(19) United States
(12) Patent Application Publication
(10) Pub. No.: US 2009/0024020 A1
(43) Pub. Date: Jan. 22, 2009

(54) AUTOMATED SYSTEM FOR INTERVENTIONAL BREAST MAGNETIC RESONANCE IMAGING

(75) Inventors: Srirama V. Swaminathan, Monmouth Junction, NJ (US); Gregory J. Metzger, Stillwater, MN (US)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 12/160,896

(22) PCT Filed: Jan. 8, 2007
(86) PCT No.: PCT/US07/60217

§ 371 Date: Jul. 15, 2008

Related U.S. Application Data
(60) Provisional application No. 60/763,477, filed on Jan. 30, 2006.

(51) Int. Cl. A61B 5/055

(52) U.S. Cl. 600/410

(57) ABSTRACT

In an interventional breast procedure, a magnetic resonance tracking sequence (80) is executed to determine (i) tracked positions of a plurality of active probe tracking coils (50) disposed with a probe (42) of an interventional instrument (40) and (ii) tracked positions of one or more active assembly tracking coils (52) disposed with a breast coil assembly (20). A probe tip position and angulation respective to the breast coil assembly is determined (84) based on the tracked positions. Conformance with a probe trajectory (88) of the determined probe tip position and angulation respective to the breast coil is verified (92).

The following relates to interventional medical procedures employing monitoring using a magnetic resonance scanner. It finds particular application in automated interventional breast magnetic resonance imaging, and will be described with particular reference thereto. It finds application more generally in conjunction with interventional procedures performed manually, semi-automatically, or fully automatically using monitoring by a magnetic resonance scanner.

Interventional breast magnetic resonance imaging employs magnetic resonance imaging during a breast biopsy or other medical procedure in which a patient's breast is penetrated by an interventional probe. In a typical approach, a breast coil at least partially surrounds the breast to provide effective electromagnetic coupling and correspondingly good magnetic resonance image quality. The breast coil includes a grid or array of openings (or a single opening configured for calibrated horizontal and vertical translation) sized to serve as guides for a perpendicularly inserted biopsy needle or other perpendicularly inserted interventional instrument probe.

One or more reference markers, such as a vitamin B capsule, that are visible to magnetic resonance imaging are inserted into one or more openings of the grid (or into the single translatable opening). The patient is then inserted into the magnetic resonance scanner, and a magnetic resonance image is acquired to identify the lesion to be probed, and its position in relation to the one or more reference markers. An appropriate opening of the grid of openings (or an appropriate position of the translatable opening) is identified for aligning the needle with the lesion, and a needle insertion distance is calculated for inserting the needle into the breast and into contact with the lesion.

The patient is then retracted from the magnetic resonance scanner and the biopsy needle is manually inserted into the appropriate opening that will serve as the needle guide for inserting the needle into the breast. The patient is moved back into the magnetic resonance scanner, and a full confirmation scan is performed to image the breast and with the marker to ensure that the needle is properly aligned. The patient is again retracted from the magnetic resonance scanner, and the needle is inserted into the breast. The needle is stabilized by the opening that serves as the needle guide, and is pressed into the breast for the calculated needle insertion distance in order to hopefully contact the lesion or other abnormality. The patient is yet again moved back into the magnetic resonance scanner, and yet another full breast image is acquired to verify that the inserted probe is in fact contacting the lesion or other abnormality. If needle contact with the lesion is confirmed, then the interventional procedure is performed. Because each imaging scan can take several minutes, the generating of multiple images can be time consuming and tedious for the patient.

In certain interventional procedures, a magnetic contrast agent is administered to the patient to provide improved imaging of the lesion or other abnormality. For example, a malignant tumor typically has its own vasculature leading to enhanced blood flow through the malignant tumor. Hence, an intravenous magnetic contrast agent that concentrates in the blood can enhance the image contrast of the malignant tumor. The intravenous contrast agent is typically taken up into the tumor faster than into other tissue, and also washes out of the tumor more quickly. This contrast agent inflow/outflow time imposes strict time constraints on the magnetic resonance imaging performed to determine needle alignment, to confirm the position of the aligned needle, and to confirm lesion contact.

Such existing interventional procedures have numerous disadvantages. They are time-consuming due in part to the repetitious long imaging scans and the repeated movement of the patient into and out of the magnetic resonance scanner. The repeated retraction of the patient from the magnetic resonance scanner in order to perform interventional instrument position adjustments, followed by insertion of the patient back into the scanner to acquire images to confirm such position adjustments, can stress the patient. Another disadvantage is that, since the needle or other interventional probe is inserted into the breast without real-time magnetic resonance monitoring, any error in needle trajectory is not discovered until after the needle is inserted. Yet another disadvantage is that the biopsy needle or other interventional probe must be inserted perpendicularly into the alignment opening that serves as the stabilizing needle guide. This geometrical constraint can make it difficult or impossible to reach inconveniently located lesions, and/or can result in an unduly long needle trajectory in the breast.

The following contemplates improvements that overcome the aforementioned limitations and others.

According to one aspect, a system is disclosed for performing an interventional breast procedure, including a magnetic resonance scanner, a probe, a breast coil assembly, and a procedure controller. A plurality of active probe tracking coils are disposed with the probe such that a position and angulation of the probe is inferable from the tracked positions of the active probe tracking coils. The breast coil assembly is configured to be disposed in an imaging region of the magnetic resonance scanner. The breast coil assembly includes one or more active assembly tracking coils disposed with the breast coil assembly such that a position of the breast coil assembly is inferable from tracked positions of the one or more active assembly tracking coils. During performance of an interventional breast procedure, the procedure controller (i) executes a magnetic resonance tracking sequence to determine tracked positions of the active probe tracking coils and one or more active assembly tracking coils, and (ii) determines a position and angulation of the probe respective to the breast coil assembly based on the tracked positions.

According to another aspect, an interventional breast procedure is disclosed. At least one magnetic resonance image of a breast is acquired using a breast coil assembly with one or more active assembly tracking coils positionally coordinated with the diagnostic image. A probe is inserted into the breast. Position and angulation of the probe are tracked at least during the inserting by iteratively executing a magnetic resonance tracking sequence to determine (i) tracked positions of a plurality of active probe tracking coils disposed with the probe and (ii) positions of the one or more active assembly tracking coils that are positionally coordinated with the diagnostic image.

According to other aspects, a system is disclosed for performing the interventional breast procedure as set forth in the preceding paragraph, and a processor is disclosed for performing the interventional breast procedure as set forth in the preceding paragraph, and a digital storage medium is disclosed encoding instructions executable to perform the interventional breast procedure as set forth in the preceding paragraph.

One advantage resides in enabling interventional breast magnetic resonance imaging procedures to be performed in shorter times.

Another advantage resides in reduced patient stress during the performance of a breast biopsy or other interventional breast procedure.

Another advantage resides in reduced likelihood of error in the insertion of the interventional instrument probe into the breast.

Numerous additional advantages and benefits will become apparent to those of ordinary skill in the art upon reading the following detailed description of the preferred embodiments.

The invention may take form in various components and arrangements of components, and in various process operations and arrangements of process operations. The drawings are only for the purpose of illustrating preferred embodiments and are not to be construed as limiting the invention.

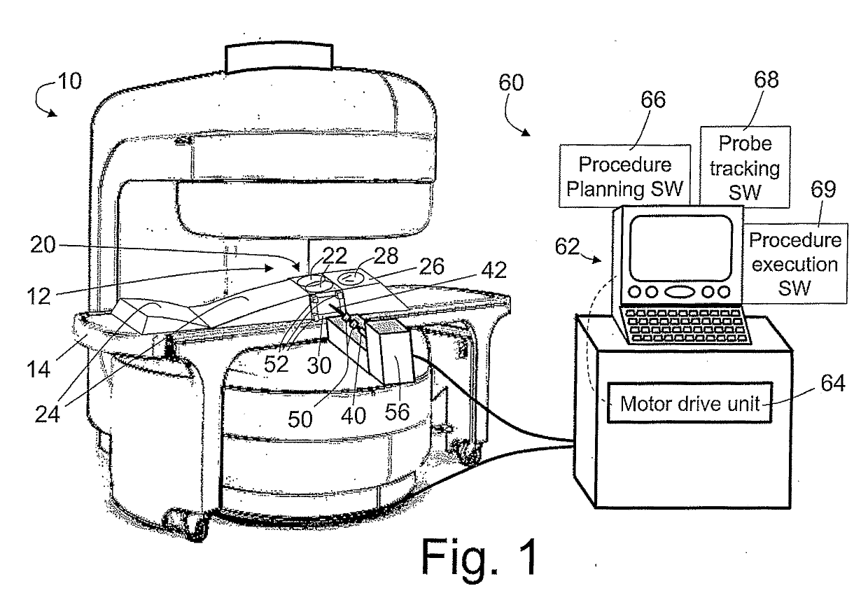

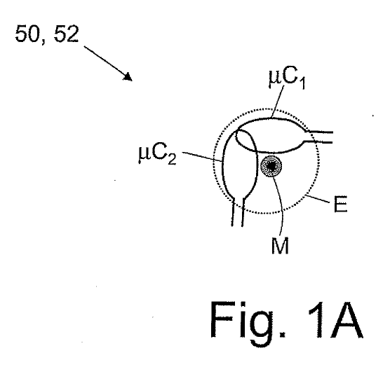

FIG. 1 diagrammatically shows an automated interventional breast magnetic resonance system.

FIG. 1A diagrammatically shows one of the active tracking coils.

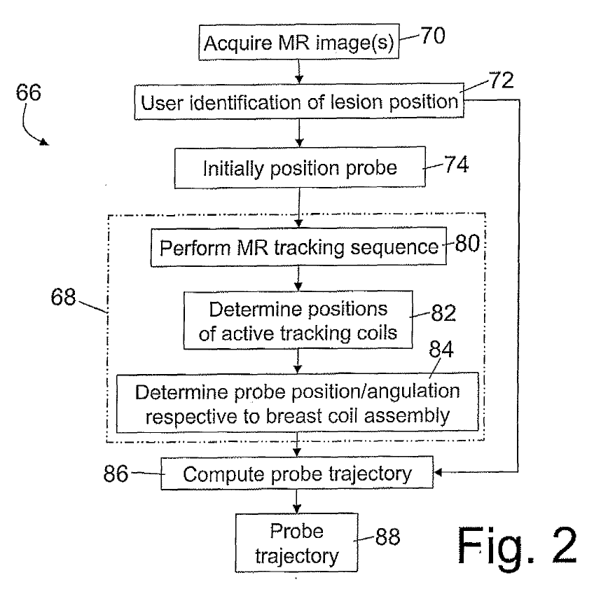

FIG. 2 shows a process sequence for determining an interventional instrument probe trajectory for use in conjunction with the interventional breast magnetic resonance system of FIG. 1

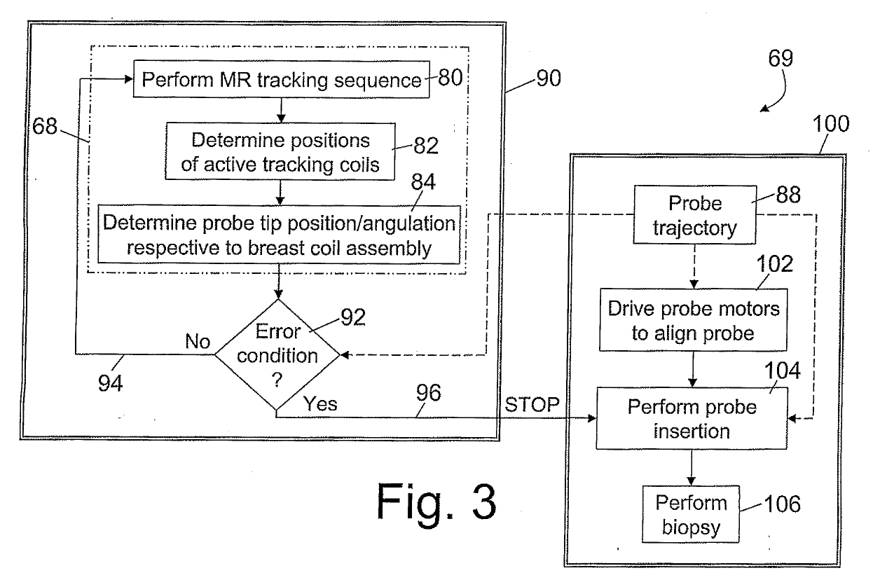

FIG. 3 shows a process sequence for performing an automated interventional breast procedure using the interventional breast magnetic resonance system of FIG. 1 and the probe trajectory determined by the process sequence shown in FIG. 2.

With reference to FIG. 1, a magnetic resonance scanner 10 performs magnetic resonance imaging in an imaging region 12. In the illustrated embodiment, the magnetic resonance imaging scanner 10, although diagrammatic, is based on a Philips Panorama 0.23T scanner available from Philips Medical Systems Nederland B.V. This scanner has an open bore that facilitates interventional medical procedures. It will be appreciated that this scanner 10 is an illustrative example, and that other types of magnetic resonance scanners can be used, including but not limited to open bore scanners, closed-bore scanners, vertical bore scanners, and so forth. Typically, the scanner will include components known in the art and hence not illustrated, such as a main magnet (superconducting or resistive) for generating a main (B₀) magnetic field in the imaging region 12, a gradient system for superimposing magnetic field gradients on the main (B₀) magnetic field in the imaging region 12, and optionally a whole-body radio frequency coil for exciting magnetic resonance in material disposed within the imaging region 12. The main magnet, magnetic field gradient system, and optional whole-body radio frequency coil are typically disposed within the housing of the magnetic resonance scanner 10 above and below, or surrounding, the imaging region, although in some embodiments certain of these components such as the whole-body radio frequency coil may be disposed on the outside of or adjacent to the housing.

A patient (not shown) who is to undergo an interventional breast procedure is placed on a subject support 14. The subject support also supports a breast coil assembly 20 including openings 22 for receiving the patient's breasts when the patient lays face-down on the subject support 14. For interventional breast magnetic resonance imaging, the subject support 14 is positioned with the breast coil assembly 20 substantially centered in the imaging region 12 of the scanner 10. In some contemplated embodiments, the breast coil assembly may be located within a recess of the subject support or otherwise physically integrated into the subject support.

The breast coil assembly 20 includes one or more radio frequency coils (not shown) positioned close to each breast inserted into the openings 22. The radio frequency coils are tunable to the magnetic resonance frequency to receive magnetic resonance signals emanating from the breasts. Typically, the radio frequency coils are single coil loops, although other types of radio frequency coils, including multi-loop radio frequency coils, can be used. In some embodiments, the radio frequency coils of the breast coil assembly 20 are used both for exciting magnetic resonance in the breasts and for receiving magnetic resonance signals. In other embodiments, a whole-body radio frequency coil (not shown) disposed in or on the housing of the scanner 10 or other coil excites the magnetic resonance in the breasts, and the radio frequency coils of the breast coil assembly 20 are receive-only coils that receive the magnetic resonance signals. In this latter arrangement, the receive-only radio frequency coils of the breast coil assembly 20 typically include electronically operable detuning circuitry to detune the receive-only radio frequency coils during the magnetic resonance excitation phase of the imaging sequence.

The illustrated breast coil assembly 20 is a dual-breast coil assembly including one or more radio frequency coils positioned close to each breast. With the dual-breast coil assembly 20 either breast can be imaged by itself, or both breasts can be imaged simultaneously. In other embodiments, the breast coil assembly may be a single-breast coil assembly with radio frequency coils coupled with only one breast (preferably the breast undergoing the interventional procedure). When using a single-breast coil assembly, the second (non-imaged) breast is suitably disposed in a passive recess similar to the recesses 22 but without breast-coupling radio frequency coils.

The patient (or at least the patients breasts) should remain stationary throughout the interventional breast procedure. Accordingly, the subject support 14 includes conformal surfaces 24 to provide adequate support for the lower torso and legs of the face-down lying patient. The head region of the patient is similarly supported by another conformal surface 26 that optionally includes a depression 28 for receiving the patient's face. The illustrated patient supports 24, 26 are examples, and other patient support arrangements or configurations can be used. In some embodiments, the patient supports are integrated with the frame of the breast coil. Optionally, the subject support 14 may further includes straps, clamps, or other patient restraints (not shown) to ensure that the patient remains stationary during throughout the interventional breast procedure. To further stabilize the breast during the interventional procedure, the breast coil 20 typically also includes compression plates 30 that compress and immobilize the breast. One compression plate is visible in the perspective view of FIG. 1, but compression plates are typically provided on either side of each breast, to compress and immobilize the breast undergoing the interventional procedure. The compression plate 30 includes an opening or array of openings providing access to the breast for performing the interventional procedure.

While the illustrated breast coil 20 and supports 24, 26 are configured to receive the patient lying face-down, it is also contemplated to employ a face-up arrangement in which the breast coil is disposed on top of the face-up lying patient and over at least the breast undergoing the interventional procedure.

An interventional instrument 40 includes a probe 42 configured to pass through the opening in the proximate compression plate 30 and to insert into a breast to perform an interventional procedure. In some embodiments, the probe 42 is a biopsy needle. However, other types of interventional instruments can be used, for example to provide targeted delivery of a drug or so forth.

A plurality of active probe tracking coils 50 are disposed with the probe 42, such as the illustrated two active probe tracking coils 50 spaced apart along the probe 42 and/or the interventional instrument 40 of which the probe 42 is a part. The active probe tracking coils are disposed with the probe 42 such that the position and angulation of the probe 42 are inferable from tracked positions of the active probe tracking coils 50. For example, the active probe tracking coils 50 are suitably disposed on or in at least one of the probe 42 and a portion of the interventional instrument 40 other than the probe 42 that has a known position relative to the probe 42. Typically, at least two active probe tracking coils 50 are used to identify both position and angulation of the probe 42.

Similarly, the one or more active assembly tracking coils 52 are disposed with the breast coil assembly 20, such as the illustrated three active assembly tracking coils 52 are disposed with the breast coil assembly 20. The active assembly tracking coils are disposed with the breast coil assembly such that the position and angulation of the breast coil assembly 20, and hence the position and angulation of the coupled breast, is inferable from tracked positions of the active assembly tracking coils 52. For example, the active assembly tracking coils 52 are suitably disposed on or in at least one of the breast coil assembly 20, the patient support 14 that supports the breast coil assembly 20, or a conformal surface 24, 26 disposed on the patient support 14 that supports the breast coil assembly 20. In some embodiments, one or more active assembly tracking coils are disposed on or in the breast that is coupled with the breast coil assembly 20. Since the breast coil assembly 20 is generally expected to be stationary during the interventional procedure, it is contemplated to employ as few as a single active assembly tracking coil 52 which would be sufficient to detect a displacement of the breast coil assembly 20 and to correlate the coordinate system of the beast and its image with the coordinate system of the breast coil/scanner/probe and the images of their active tracking coils 50, 52. To more accurately determine the position of the breast coil assembly 20, additional active assembly tracking coils can be used. The illustrated three non-collinear active assembly tracking coils 52 are sufficient to identify the position and any rotation of the breast coil assembly 20.

With brief reference to FIG. 1A, each tracking coil 50, 52 suitably includes a vial of marker material M and one or more microcoils, such as the illustrated two orthogonally oriented single-loop microcoils μC1, μC2, coupled with the marker material M to detect magnetic resonance emanating from the marker material. Optionally, the vial of marker material M and the one or more microcoils μC1, μC2 are encased in an encapsulating epoxy E or otherwise housed or contained. Tracking is performed using a magnetic resonance tracking sequence executed by the magnetic resonance scanner 10. A suitable magnetic resonance tracking sequence may include, for example: (i) a spatially non-selective radio frequency excitation pulse that excites magnetic resonance in the marker material of the active tracking coils 50, 52; and (ii) a plurality of one-dimensional projection readouts. During each one-dimensional projection readout, the one or more microcoils of each tracking coil 50, 52 generate readout data that enables localizing of the corresponding marker material along the projection direction. By performing such one-dimensional projection readouts along a plurality of different directions, the position of each tracking coil 50, 52 in three-dimensional space is determined. Due to the strength of the signals, as few as three orthogonal projections can be sufficient. Fewer projections may be sufficient if other positional constraints are known. Such projections can be acquired tens or hundreds of times per second.

With returning reference to FIG. 1, for performing an automated interventional procedure, an optional motorized drive 56 is provided for manipulating the probe 42 in accordance with a pre-determined probe trajectory in order to bring the probe 42 (i.e., at least a tip of the probe) into contact with a lesion or other feature to be probed or otherwise interventionally processed. (As used herein, “lesion” is to be broadly construed as encompassing any feature that is the target of the interventional procedure. For biopsy procedures, the lesion is typically an abnormal growth or tumor that is suspected of being cancerous. However, the lesion can be another type of feature.) In some embodiments, the motorized drive 56 is a pneumatic cylinder or motor which is made of magnetic field-compatible materials and is disposed within a main magnetic field generated by the magnetic resonance scanner 10. In other embodiments, the motorized drive is not magnetic field-compatible, and accordingly is disposed outside of the main magnetic field and mechanically coupled with the interventional instrument 40 via a magnetic field-compatible arm or other connector. In some embodiments, the probe 42 is moved manually, in which case the motorized drive 56 is replaced by suitable user controls (not shown) that can be manipulated by the medical doctor or other qualified medical person to perform the interventional procedure manually.

A procedure controller 60 includes a user interface 62, such as the illustrated personal computer 62, or laptop computer, network computer, handheld controller with keypad and LCD display, a scanner control unit, or so forth. The procedure controller includes an optional motor drive unit 64 that is provided to control the motorized drive 56 if the motorized drive 56 is provided for performing automated interventional procedures. A procedure planner 66, implemented in the illustrated embodiment as procedure planning software 66 executing on the user interface 62, or in other embodiments implemented as a separate processing unit, computes a probe trajectory for aligning the probe 42 with a lesion or other feature of interest and for inserting the probe into the breast and into contact with the lesion of interest. A probe tracker 68, implemented in the illustrated embodiment as probe tracking software 68 executing on the user interface 62, or in other embodiments implemented as a separate probe tracking processor, causes the magnetic resonance scanner 10 to perform the magnetic resonance tracking sequence and determines the position and angulation of the probe 42 respective to the breast coil assembly 20 (or, equivalently, respective to the breast contained in the breast coil assembly 20). Procedure execution software 69 performs monitoring of the probe 42 (in conjunction with the probe tracking software 68) to ensure that the probe 42 follows the planned probe trajectory. In some embodiments, a diagrammatic representation of the trajectory and the probe (or probe tip) position calculated by the tracking software 68 is superimposed on a diagnostic image displayed on the user interface 62. In embodiments in which manual probe insertion is used, the surgeon watches the display portion of the user interface 62 to observe the substantially real time display of the probe trajectory and probe (or probe tip) position. In embodiments in which the probe 42 is automatically inserted, the procedure execution software 69 also controls the motor drive unit 64 to operate the motorized drive 56 to manipulate the probe 42 in accordance with the probe trajectory.

With continuing reference to FIG. 1 and with further reference to FIG. 2, an example embodiment process suitably performed by the procedure planning software 66 is described. One or more initial magnetic resonance images are acquired in a process operation 70, and the acquired image or images are displayed on the user interface 62. The user identifies a position of a lesion or other feature of interest in a process operation 72. The user interface 62 is configured to receive the user indication of the position of the lesion in the displayed magnetic resonance image, for example using a mouse pointer or other pointer device interacting with axial, coronal, and sagittal slice representations of the breast. Optionally, the user also identifies a position of one or more of the active assembly tracking coils 52 to positionally coordinate the active assembly tracking coils 52 with the one or more magnetic resonance images. Alternatively, the active assembly tracking coils 52 can be positionally coordinated with the images based on the common use of the magnetic resonance scanner 10 in performing both imaging and tracking. The probe 42 is initially positioned in a process operation 74. (This initial positioning 74 can optionally be performed before the acquisition of the magnetic resonance image or images in the process operation 70).

The probe tracking software 68 is invoked to determine the initial position and angulation of the probe 42 respective to the breast coil 20. In a suitable tracking process, the magnetic resonance tracking sequence (for example, including a spatially non-selective radio frequency excitation pulse followed by a plurality of one-dimensional projection readouts employing the microcoils of the tracking coils 50, 52 as magnetic resonance receivers) is executed by the magnetic resonance scanner 10 in a process operation 80. The position of each active tracking coil 50, 52 is determined based on readouts acquired by the one or more microcoils of that tracking coil during the magnetic resonance tracking sequence in a process operation 82. The position and angulation of the probe 42 respective to the breast coil assembly 20 is inferred from the determined positions of the active tracking coils 50, 52 based on a known spatial relationship of the probe 42 and its tip respective to the active probe tracking coils 50, and further based on a known spatial relationship of the breast coil assembly 20 respective to the active assembly tracking coils 52, in a process operation 84.

With the initial position and angulation of the probe 42 and the position of the lesion both known in a common coordinate system (for example, using the position of the breast coil assembly 20 as a common reference), a probe trajectory 88 is computed in a process operation 86 for (i) moving the probe 42 (with the probe 42 disposed outside of the breast) to an aligned position and angulation in which the position of the lesion lies along a direction defined by the aligned probe 42, and (ii) for translating the aligned probe 42 along the direction defined by the aligned probe 42 to bring at least the tip of the probe 42 into contact with the lesion.

In some embodiments, the probe 42 may have a fixed angulation (for example, perpendicular to the face of the compression plate 30), in which case the probe trajectory 88 is limited to translation operations. The alignment portion of the probe trajectory 88 suitably includes translating the probe 42 in a direction transverse to the direction of the probe. In these embodiments, the tracking 68 optionally expressly tracks only the tip position (including the probe tip insertion distance), and not probe angulation, since the probe angulation is assumed to be fixed. However, the probe angulation is optionally actively tracked by the tracking 68 even if the angulation is nominally fixed, in order to detect potential problems such as tilting, bending, or flexing of the probe 42.

In other embodiments, the probe 42 includes an adjustable angulation controlled, for example, by the motorized drive 56. In these embodiments, the alignment portion of the probe trajectory 88 optionally includes adjusting an angulation of the probe 42 to point the probe 42 toward the position of the lesion of interest, and the tracking 68 actively tracks both position and angulation of the probe 42 to ensure that the angulation adjustments are properly made in conformance with the probe trajectory 88.

With continuing reference to FIG. 1 and with further reference to FIG. 3, an exemplary process embodiment suitably performed by the procedure execution software 69 is described. During execution of the interventional procedure in accordance with the probe trajectory 88, a monitoring portion 90 of the procedure execution software 69 employs the probe tracking software 68 (for example, by performing the tracking operations 80, 82, 84 as shown in FIG. 3) to track the position and angulation of the probe 42 respective to the breast coil assembly 20. An error condition checker 92 compares the probe tip position and probe angulation with the probe trajectory 88. As long as the probe tip position and angulation conforms with the probe trajectory 88 (typically to within selected tolerances) the error condition checker 92 iterates 94 execution of the probe tracking software 68 to perform a probe tip position and angulation check at selected intervals, for example five times per second, ten times per second, one-hundred times per second, every millimeter of projected probe movement, or so forth. In some embodiments, the probe tip position and angulation are checked sufficiently frequently that a diagrammatic probe tip position and angulation superimposed on a diagrammatic image shown on the user interface 62 is updated substantially in real time and the movement appears smooth and continuous to the surgeon or other observer.

If the error condition checker 92 finds that the probe tip position and/or probe angulation has deviated beyond the selected tolerances from the probe trajectory 88, then a suitable remedial action 96 is performed. In the illustrated embodiment, the remedial action is to stop the interventional procedure; however, additional or alternative remedial actions may be taken. If the probe 42 is being manipulated manually, then a suitable remedial action may be to display a prominent visual warning, with an optional accompanying audio warning, to warn the medical doctor or other actor that the probe 42 has deviated from the probe trajectory 88. If the doctor then adjusts the probe 42 into conformance with the probe trajectory 88, then the visual and optional audio warnings are suitably turned off.

In the illustrated embodiment, the probe 42 is automatically manipulated using the motorized drive 56 and motor drive unit 64 operated by an optional automatic probe manipulation portion 100 of the procedure execution software 69. For example, the probe motors are suitably driven in a process operation 102 to align the probe 42 with the lesion in accordance with a probe alignment portion of the probe trajectory 88, followed by inserting the probe into the breast and into contact with the lesion in a process operation 104. At least during the probe insertion operation 104, and optionally also during the probe alignment operation 102, the monitoring portion 90 of the procedure execution software 69 is active to monitor the probe tip position and angulation. If the monitored probe tip position and/or probe angulation deviates beyond selected tolerances from the probe trajectory 88, then the error condition checker 92 suitably detects such deviation and sends a “STOP” signal as at least a portion of the remedial action 96 to stop further automated movement of the probe 42.

Once the probe trajectory 88 has been followed to completion, the monitoring portion 90 of the procedure execution software 69 (or the medical doctor) suitably verifies that the probe tip has contacted the lesion of interest. After such confirmation, the biopsy or other interventional procedure is performed in a process operation 106. After the interventional procedure is complete, the probe 42 is suitably withdrawn by repeating the probe insertion operation 104 with the direction of probe movement reversed to withdraw the probe 42 from the breast. The probe withdrawal is optionally also monitored by the monitoring portion 90 of the procedure execution software 69.

Although not illustrated, the procedure execution software 69 optionally includes imaging sequences performed by the magnetic resonance scanner 10, suitably interleaved between iterations of the magnetic resonance tracking sequence process operation 80, to provide real time images of the probe 42 as it is aligned and enters the breast. Between full imaging sequences, the current probe trajectory and tip position are suitably diagrammatically displayed superimposed on the breast image. This enables the medical professional to monitor the current trajectory and tip position substantially in real time on the user interface 62. The medical professional can stop or override the procedure, if appropriate. However, since the monitoring portion 90 of the procedure execution software 69 performs automated verification of conformance with the probe trajectory 88 during probe insertion, such imaging is optionally omitted.

In the illustrated embodiment, the procedure planning software 66, probe tracking software 68, and procedure execution software 69 are executed on one or more processors of the user interface 62. In other embodiments, the processor used to execute some or all of this software may be a separate dedicated processor disposed with the magnetic resonance scanner 10, or a separate processor disposed on a digital network accessed by the user interface 62, or so forth. Some or all of the software 66, 68, 69 may be stored on a digital storage medium or media such as a magnetic disk, an optical disk, electronic random access memory (RAM), electronic read-only memory (ROM), non-volatile or battery-backed electronic read-write memory such as an EPROM, EEPROM, FLASH memory, or so forth.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1. A system for performing an interventional breast procedure, the system comprising:
a magnetic resonance scanner;
a probe and a plurality of active probe tracking coils disposed with the probe such that a position and angulation of the probe is inferable from the tracked positions of the active probe tracking coils;
a breast coil assembly configured to be disposed in an imaging region of the magnetic resonance scanner, the breast coil assembly including one or more active assembly tracking coils disposed with the breast coil assembly such that a position of the breast coil assembly is inferable from tracked positions of the one or more active assembly tracking coils; and
a procedure controller which during performance of an interventional breast procedure (i) executes a magnetic resonance tracking sequence to determine tracked positions of the active probe tracking coils and one or more active assembly tracking coils, and (ii) determines position and angulation of the probe respective to the breast coil assembly based on the tracked positions.

2. The system as set forth in claim 1, wherein the procedure controller further (iii) verifies conformance with a probe trajectory of the determined position and angulation of the probe respective to the breast coil.

3. The system as set forth in claim 1, wherein the one or more active assembly tracking coils include at least three non-collinear active assembly tracking coils.

4. The system as set forth in claim 1, wherein the magnetic resonance tracking sequence includes:
a spatially non-selective radio frequency excitation pulse; and
a plurality of one-dimensional projection readouts.

5. The system as set forth in claim 1, wherein:
the plurality of active probe tip tracking coils are disposed on or in at least one of (i) the probe, and (ii) an interventional instrument of which the probe is a part; and
the one or more active assembly tracking coils are disposed on or in at least one of (i) the breast coil assembly, (ii) a patient support that supports the breast coil assembly, (iii) a conformal surface disposed on a patient support that supports the breast coil assembly, and (iv) a breast coupled with the breast coil assembly.

6. The system as set forth in claim 1, wherein the procedure controller includes:
a user interface for displaying one or more magnetic resonance images acquired by the magnetic resonance scanner, the user interface being configured to receive a user indication of a position of a lesion in the displayed one or more magnetic resonance images.

7. The system as set forth in claim 6, wherein the procedure controller further (iii) superimposes an indication of the determined position and angulation of the probe respective to the breast coil assembly on a diagnostic image displayed on the user interface.

8. The system as set forth in claim 7, wherein the procedure controller iterates the executing (i) of the magnetic resonance tracking sequence, the determining (ii) of the position and angulation of the probe, and the superimposing (iii) of the indication of the determined position and angulation of the probe to provide a substantially real time visual indication of the position and angulation of the probe.

9. The system as set forth in claim 1, wherein the procedure controller includes:
procedure planning software configured to compute a probe trajectory for moving the probe into contact with a lesion based on (i) the currently determined probe position and angulation and (ii) a determined position of the lesion.

10. The system as set forth in claim 9, wherein the computed probe trajectory includes (i) an alignment portion setting forth movement of the probe with the probe disposed outside of the breast to an aligned position and angulation in which the position of the lesion lies along a direction defined by the aligned probe, and (ii) an insertion portion setting forth translation of the aligned probe to bring the probe into contact with the lesion.

11. The system as set forth in claim 9, further including:
a motorized drive for manipulating the probe in accordance with the computed probe trajectory in order to bring the probe into contact with the lesion.

12. The system as set forth in claim 11, wherein the motorized drive is made of magnetic field-compatible materials and is disposed within a main magnetic field generated by the magnetic resonance scanner.

13. The system as set forth in claim 1, wherein the probe is a biopsy needle.

14. An interventional breast procedure comprising:
acquiring at least one magnetic resonance image of a breast using a breast coil assembly with one or more active assembly tracking coils positionally coordinated with the diagnostic image;
inserting a probe into the breast; and
tracking position and angulation of the probe at least during the inserting by iteratively executing a magnetic resonance tracking sequence to determine (i) tracked positions of a plurality of active probe tracking coils disposed with the probe and (ii) positions of the one or more active assembly tracking coils that are positionally coordinated with the diagnostic image.

15. The interventional breast procedure as set forth in claim 14, further including:
during the inserting, verifying conformance with a probe trajectory of the tracked position and angulation of the probe.

16. The interventional breast procedure as set forth in claim 15, wherein the inserting includes:
operating a motorized drive to manipulate the probe in accordance with the probe trajectory.

17. The interventional breast procedure as set forth in claim 15, further including:
computing the probe trajectory based on (i) an initial position and angulation of the probe determined by at least one iteration of the executing, determining, and verifying and (ii) a position of a lesion to be probed determined based on the at least one magnetic resonance image.

18. The interventional breast procedure as set forth in claim 14, further including:
superimposing the tracked position and angulation of the probe on the diagnostic image to provide a substantially real time visual indication of the tracked position and angulation of the probe.

19. A system for performing the interventional breast procedure as set forth in claim 14.

20. A digital storage medium encoding instructions executable to perform the interventional breast procedure as set forth in claim 14.

21. A processor programmed to perform the interventional breast procedure as set forth in claim 14.
